(19) United States
(12) Patent Application Publication
(10) Pub. No.: US 2003/0219451 A1
(43) Pub. Date: Nov. 27, 2003

(54) Stable helical C peptides and uses therefor

(75) Inventors: Samuel K. Sia, Cambridge, MA (US); Peter S. Kim, Bryn Mawr, PA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Whitehead Institute for Biomedical Research, Cambridge, MA

(21) Appl. No.: 10/283,403

(22) Filed: Oct. 29, 2002

Related U.S. Application Data
(60) Provisional application No. 60/334,528, filed on Nov. 30, 2001.
(60) Provisional application No. 60/350,099, filed on Oct. 29, 2001.

(51) Int. Cl. A61K 39/21; C07K 14/16

(52) U.S. Cl. 424/188.1; 530/350

(57) ABSTRACT

Stable, helical, biologically active peptides, particularly stable, helical, biologically active C-peptides, which comprise all or a portion of an unstructured peptide (a peptide which is unstructured in solution) linked to a scaffold (or support) polypeptide. Such peptides are referred to herein as structured C-peptides. In particular embodiments, structured C-peptides of the present invention comprise all or a portion of a binding epitope of a C-peptide, such as all or a portion of a binding epitope of a peptide derived from the carboxy-terminal region of the HIV-1 gp41 envelope glycoprotein. The C-peptide can be, for example, C34 peptide of HIV-1 gp41.

### RELATED APPLICATION(S)

This application claims the benefit of U.S. Provisional Application No. 60/334,528, filed Nov. 30, 2001 and U.S. Provisional Application No. 60/350,099, filed Oct. 29, 2001.

The entire teachings of the above application(s) are incorporated herein by reference.

### GOVERNMENT SUPPORT

The invention was supported, in whole or in part, by a grant GM44163 from the National Institutes of Health. The Government has certain rights in the invention.

### BACKGROUND OF THE INVENTION

Infection of cells by HIV-1 requires fusion of the viral and cellular membranes—a process mediated by the viral envelope glycoprotein complex (gp120/gp41) and receptors on the target cell (CD4 and a chemokine co-receptor such as CCR-5 or CXCR-4). Recent studies on the HIV-1 membrane fusion process raise hopes for the development of drugs against HIV-1 entry. Such fusion inhibitors will complement the existing anti-HIV-1 combination therapies that target HIV-1 protease and reverse transcriptase.

A promising class of fusion inhibitors is peptides derived from the carboxy-terminal α-helical domain of gp41 (referred to as C-peptides), such as C34 and T-20. These peptides are potent (nanomolar) inhibitors of HIV-1 infection and syncytia formation in cell culture experiments. Data from clinical trials show the antiviral activity of T-20 in humans. Substantial evidence indicates that C-peptides, unstructured in solution, bind in a helical manner to the coiled-coil amino-terminal (N-peptide) region of a transiently exposed conformation of gp41. This binding event prevents the formation of the trimer-of-hairpins structure, which is necessary for the fusion of the viral and cellular membranes.

Although they are effective HIV-1 entry inhibitors, C-peptides are sensitive to proteolytic degradation due to their lack of secondary and tertiary structures in solution. This sensitivity may contribute to poorer pharmacokinetics in vivo. For example, T-20 shows potent antiviral activity in humans, but a large dose (200 mg/day) of the peptide is necessary for efficacy. This dose is 10³-fold higher than the IC₅₀ for T-20 in cell culture. It would be helpful to have a means by which unstructured peptides, such as the C-peptides, can be made proteases resistant while maintaining their biological activity.

### SUMMARY OF THE INVENTION

Described herein are structured C-peptides comprising all or a portion of a C-peptide (e.g., from a virus) and a polypeptide scaffold. In particular, described herein are stable, helical, biologically active peptides, particularly stable, helical, biologically active C-peptides, which comprise all or a portion of an unstructured peptide (a peptide which is unstructured in solution) linked to a scaffold (or support) polypeptide. Such peptides are referred to herein as structured C-peptides. In particular embodiments, structured C-peptides of the present invention comprise all or a portion of a binding epitope of a C-peptide, such as all or a portion of a binding epitope of a C-peptide derived from a virus (e.g., the carboxy-terminal heptad repeat region of the HIV-1 gp41 envelope glycoprotein) and a polypeptide scaffold. The C-peptide can be, for example, C34 peptide of HIV-1 gp41 (Chan, D. and Kim, P. S., Cell, 93:681-684 (1998)). Alternatively, all or a portion of a binding epitope of a peptide derived from the carboxy terminal heptad repeat region of the HIV-1 gp41 envelope glycoprotein, such as all or a portion of C34 peptide of HIV-1 gp41 and all or a portion of the functional epitope on HIV-1 gp41 for the anti-gp41 human monoclonal antibody 2F5, are linked to a scaffold (or support) polypeptide. In addition, helical C-peptides of other viruses, such as respiratory syncytial virus, ebola virus, Simian Immunodeficiency Virus (SIV), parainfluenza virus (SV5), and influenza virus, can be included in a structured C-peptide of the present invention. All or a portion of a selected helical C-peptide, such as all or a portion of the large binding epitope of HIV-1 gp41 C34, can be used. It is not necessary that the entire epitope is used; a portion that includes an appropriate amino acid composition (number and identity of amino acid residues) such that it retains function (e.g., its activity as an inhibitor of HIV-1 or its ability to cause production of or raise antibodies that neutralize HIV infection) can be linked to a scaffold to produce a structured C-peptide.

A specific embodiment of the structured C-peptide of the present invention comprises amino acid residues of HIV gp41 C34 peptide sufficient to produce a stable, helical HIV inhibitor when linked to an appropriate polypeptide scaffold, such as all or a portion of the GCN4 leucine zipper (e.g., the solvent-exposed face of the GCN4 leucine zipper). A particular embodiment of structured C-peptides of the present invention is denoted C34-GCN4 heterodimer, also referred to as “C34-GCN4 structured C-peptide” or “C34coil”.

In particular, the present invention relates to a structured C-peptide comprising all or a portion of a C-peptide from a virus and a polypeptide scaffold (e.g., C34-GCN4). The structured C-peptide can further comprise a second polypeptide scaffold, and the second polypeptide scaffold can be the same as, or different from, the first polypeptide scaffold (e.g., C34-GCN4 heterodimer or C34coil). In one embodiment, the structured C-peptide comprises all or a portion of HIV-1 gp41 C34 peptide and a polypeptide scaffold which is a GCN4 leucine zipper or a portion thereof. In another embodiment, the structured C-peptide comprises a binding epitope of HIV-1 C34 linked to a GCN4 leucine zipper, wherein the structured C-peptide is stable, helical and biologically active. The structured C-peptide can further comprises a second GCN4 leucine zipper linked to the structured C-peptide (e.g., via a disulfide bond between two cysteine residues).

The present invention also relates to a structured C-peptide comprising a first peptide and a second peptide, the first peptide comprising all or a portion of a C-peptide from a virus (e.g., a C-peptide from a virus is a binding epitope of HIV-1 C34) grafted onto the surface of a GCN4 leucine zipper and the second peptide comprising an additional GCN4 peptide, wherein the first and second peptide are linked and the structured C-peptide is stable, helical and biologically active. In a particular embodiment, the present invention relates to a structured C-peptide comprising a first peptide and a second peptide, the first peptide comprising all or a portion of a binding epitope of HIV-1 C34 grafted onto the surface of a GCN4 leucine zipper and the second peptide comprising an additional GCN4 peptide, wherein the first and second peptide are linked and the structured C-peptide is stable, helical and biologically active. In another embodiment, the invention relates to a structured C-peptide comprising a first peptide and a second peptide, the first peptide comprising all or a portion of a binding epitope of HIV-1 C34 grafted onto the surface of a GCN4 leucine zipper and the second peptide comprising an additional GCN4 peptide, wherein the first and second peptide are linked, Asn 16 in each GCN4 peptide is replaced with Lys, and the structured C-peptide is stable, helical and biologically active (e.g., C34-GCN4-N16K).

The present invention also relates to a method of producing a structured viral C-peptide that is stable, helical and biologically active comprising grafting a first peptide comprising all or a portion of a C-peptide from a virus onto the surface of a polypeptide scaffold (e.g., GCN4 leucine zipper); and linking a second peptide comprising an additional polypeptide scaffold (e.g., GCN4 leucine zipper) to the first peptide, thereby producing a structured viral C-peptide that is stable, helical and biologically active.

The present invention also relates to a method of eliciting an immune response in an individual, comprising introducing into the individual a structured C-peptide. In one embodiment, the present invention relates to a method of eliciting an immune response to a virus in an individual, comprising introducing into the individual a structured C-peptide comprising all or a portion of a C-peptide from a virus and a polypeptide scaffold. In another embodiment, the present invention relates to a method of eliciting an immune response to HIV in an individual, comprising introducing into the individual a structured C-peptide comprising a binding epitope of HIV-1 C34 linked to a GCN4 leucine zipper, wherein the structured C-peptide is stable, helical and biologically active. The structured C-peptide for use in the method can comprise, for example, a first peptide and a second peptide, the first peptide comprising all or a portion of a binding epitope of HIV-1 C34 grafted onto the surface of a GCN4 leucine zipper and the second peptide comprising an additional GCN4 leucine zipper, wherein the first and second peptide are linked and the structured C-peptide is stable, helical and biologically active.

The present invention also relates to a method of inhibiting entry of HIV into cells of an individual comprising introducing into the individual a structured C-peptide comprising a first peptide and a second peptide, the first peptide comprising all or a portion of a binding epitope of HIV-1 C34 grafted onto the surface of a GCN4 leucine zipper and the second peptide comprising an additional GCN4 leucine zipper, wherein the first and second peptide are linked and the structured C-peptide is stable, helical and biologically active.

The present invention also relates to a method of identifying a drug that binds to an amino terminal region of HIV gp41 comprising combining the amino-terminal region of gp41, a structured C-peptide comprising a first peptide and a second peptide, the first peptide comprising all or a portion of a binding epitope of HIV-1 C34 grafted onto the surface of a GCN4 leucine zipper and the second peptide comprising an additional GCN4 leucine zipper, wherein the first and second peptide are linked and the structured C-peptide is stable, helical and biologically active, and a candidate drug thereby producing a combination. The combination is maintained under conditions appropriate for binding of the structured C-peptide to the amino terminal region of gp41, and the extent to which the structured C-peptide binds to the amino terminal region of gp41 in the presence of the candidate drug is determined and compared to a control sample. If the binding of the structured C-peptide to the amino terminal region of gp41 occurs to a lesser extent in the presence of the candidate drug compared to a control sample, then the drug binds to an amino terminal region of HIV gp41.

Thus, structured C-peptides of the present invention are useful to inhibit HIV-1 entry into cells, such as human cells, and can be used to reduce (partially or totally) infection of cells by HIV-1. They are useful, for example, as vaccines to protect humans against HIV infection. They can be administered to an individual (particularly a human) in need of protection against infection by HIV and are useful as therapeutic peptides (to block entry of HIV into cells) and/or as vaccines (to elicit an immune response to an HIV epitope, such as all or a portion of the large binding epitope of C34). Structured C-peptides of the present invention are also useful in identifying a drug that binds to an amino terminal region of a virus (e.g., HIV).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C show the design of C34coil.

FIG. 1A (Left) shows the six-helix bundle of HIV-1 gp41 with one C34 helix in red/orange. FIG. 1A (Middle) shows the dimeric GCN4 leucine zipper with one helix in blue. FIG. 1A (Right) shows the model of C34coil. The critical residues of the binding epitope of C34 (orange) and the critical residues of the GCN4 hydrophobic core (blue) are incorporated into C34coil. The main-chain atoms of C34 and GCN4 superimpose to about 1 Å.

FIG. 1B shows helical wheel representations of C34 (red), GCN4 (blue) and C34coil (red and blue). C34coil is a covalent heterodimer of two peptides, (Cys)C34-GCN4 and (Cys)GCN4, connected via a disulfide bond ((Cys) denotes the addition of a Cys-Gly-Gly sequence to the N-terminus of the peptide). The boxed residues of C34 and GCN4 are incorporated into the C34-GCN4 peptide, and are equivalent to the residues highlighted in part A. Note that the four residues at the g′ position of C34 are identical to the four residues at the e position of GCN4.

FIG. 1C shows the sequences of the peptides GCN4 (SEQ ID NO: 1), C34 (SEQ ID NO: 2), (Cys)C34-GCN4 (SEQ ID NO: 3) and CGG-GCN4 (SEQ ID NO: 4), CGG-C34-GCN4 (SEQ ID NO: 5),CGG-GCN4-N16K (SEQ ID NO: 6), CGG-C34-GCN4-N16K (SEQ ID NO: 7), CGG-GCN4-pIL (SEQ ID NO: 8) and CGG-C34-GCN4-pIL (SEQ ID NO: 9) The C34-GCN4 peptide, which lacks the N-terminal Cys-Gly-Gly, was also synthesized. “Ac” and “NH2” denote an acetylated N-terminus and an amidated C-terminus, respectively. Note that the GCN4 sequence used in this study corresponds to GCN4-p1 in previous studies (E. K. O'Shea, J. D. Klemm, P. S. Kim, T. Alber, Science 254, 539-544 (1991)).

FIGS. 2A-2E are graphs showing secondary structure, stability and oligomeric state of C34coil.

FIG. 2A is two graphs showing the CD spectrum of C34, C34-GCN4 and C34coil. The guanidine hydrochloride used to solubilize C34-GCN4 absorbs light at wavelengths below 210 nm. Experimental conditions were PBS, pH 7.0, 25° C. C34coil exhibits a helical structure ([Θ]₂₂₂ value of −29,400 deg cm² dmol⁻¹), whereas C34 (−3,800 deg cm² dmol⁻¹) and the C34-GCN4 peptide (−12,400 deg cm² dmol⁻¹) exhibit random coil conformations.

FIG. 2B is two graphs showing guanidine hydrochloride denaturation of C34coil, as monitored by CD spectroscopy at 222 nm. The fitted line to a two-state unfolding transition is shown as a black line. C34coil unfolds at a midpoint of 3.6 M guanidine hydrochloride, with a free energy of unfolding of 9.3 kcal/mol.

FIG. 2C is a graph showing gel filtration chromatography of C34coil, as monitored by absorbance at 280 nm. Shown are C34coil and a buffer blank. The elution times of molecular weight standards (in kilodaltons) are indicated. C34coil exhibits an apparent molecular weight of 7000±2000 Da (expected molecular weight of 8740 Da for a monomer), with no detectable aggregation.

FIG. 2D is a graph of [heterodimer] (nM) versus Number of Syncytia.

FIG. 2E is a graph of [heterodimer] (nM) versus RLU.

FIGS. 3A-3B are graphs showing inhibition of HIV-1 envelope mediated membrane fusion. The data represent the mean±standard error of at least two separate experiments.

FIG. 3A is a graph showing inhibition of cell-cell fusion. The peptides were tested for inhibiting the fusion of HIV-1 Env expressing cells (CHO gp 160) with CD4expressing cells (CD4 HeLa) (D. C. Chan, et al., Proc. Natl. Acad. Sci., 95:15613-15617 (1998)). The IC₅₀ values for C34coil and C34-GCN4 are 3.1±0.8 nM and 4.6±0.9 nM, respectively. The (Cys)GCN4-homodimer shows no inhibitory activity up to 50 μM.

FIG. 3B is a graph showing inhibition of viral infectivity. The peptides were tested for inhibition of CD4 positive target cells (HOST4) by recombinant, luciferase-expressing HIV-1 (D. C. Chan, C. T. Chutkowski, P. S. Kim, Proc. Natl. Acad. Sci. 95, 15613-15617 (1998)). The IC₅₀ values for C34 coil and C34-GCN4 are 16±2 and 19±3 nM, respectively. The (Cys)GCN4-homodimer shows no inhibitory activity up to 50 μM.

FIGS. 4A-4B are graphs showing the sensitivity to proteolytic degradation of C34coil and C34. Peptides are incubated with proteinase K at 37° C., and the reaction products are monitored by reverse-phase HPLC. The relative ratios of proteinase K account for differences in both the protease concentrations and incubation times. Shown are the HPLC chromatograms. C34coil is approximately 1000-fold more resistant to degradation by proteinase K than C34.

FIG. 5 is a schematic representation of considerations relating to C34-GCN4 as a vaccine candidate.

FIG. 6 is a schematic representation of examples of viruses with helical C-peptides, for which structured C-peptides of the present invention may be useful.

### DETAILED DESCRIPTION OF THE INVENTION

Described herein are structured C-peptides comprising all or a portion of a C-peptide (e.g., from a virus) and a polypeptide scaffold. In particular embodiments, the structured C-peptides are designed to have a stable helical structure; be conformationally flexible to permit them to adopt an active conformation; act as a potent inhibitor of a virus (such as HIV-1); and have improved resistance to proteolytic degradation. Structured C-peptides of the present invention comprise a C-peptide from a virus, such as HIV-1 gp41 C34 peptide, linked to a polypeptide scaffold, such as the GCN4 leucine zipper. In particular embodiments, the structured C-peptides are stable, helical and biologically active forms of the viral C-peptide which have enhanced resistance to proteolytic degradation (relative to that of the viral C-peptide that is not linked to the polypeptide scaffold). The following discussion exemplifies the present invention with reference to HIV-1 gp41 C34 peptide, but it is to be understood that the present invention is applicable to other viruses with helical C-peptides.

Peptides derived from the carboxy-terminal region of the HIV-1 gp41 envelope glycoprotein (C-peptides) are potent inhibitors of HIV-1 entry. Such C-peptides are referred to herein as viral C-peptides or C-peptides derived from a virus or a viral region, indicating that the amino acid sequences of such peptides are derived from or based on the sequence of a viral region. Their mechanism of inhibition involves binding in a helical conformation to the amino-terminal region of gp41, thereby blocking the conformational change necessary to mediate fusion of the viral and cellular membranes. Because C-peptides are unstructured when not bound to gp41, they are sensitive to proteolytic degradation, which limits their potency in vivo. As described herein, Applicants have designed and implemented a method for stabilizing C-peptides, such as antifusion HIV-1 gp41 C-peptides currently in clinical trials. To enhance resistance to proteolytic degradation of C-peptides of HIV-1 gp41, the complete binding epitope of an HIV-1 gp41 C-peptide (19 amino acids) was grafted onto (linked to) a stable protein scaffold. In one embodiment, the C-peptide was grafted onto the outside face of the GCN4 leucine zipper, thereby generating a C34-GCN4 peptide. The C34-GCN peptide is very hydrophobic and unable to form a stable, helical coiled coil structure. However, the C34-GCN4 peptide is a strong inhibitor of HIV-1 entry into cells.

In another embodiment of the present invention, the C34-GCN4 peptide was linked to an additional (a second) polypeptide scaffold such as an additional GCN4 leucine zipper, thereby producing a C34-GCN4 heterodimer. The resulting heterodimer, denoted “C34-GCN4 heterodimer” or “C34coil”, is remarkably well-structured in solution and resistant to proteolytic degradation. Assessment of C34-GCN4 heterodimer demonstrated that it is a potent inhibitor of HIV-1 entry into cells. Even with 40% of its residues mutated, C34-GCN4 shows nanomolar inhibition of HIV-1 entry. The C34-GCN4 peptide can be linked to a second polypeptide scaffold (e.g., GCN4 leucine zipper) using a variety of methods. In one embodiment, the C34-GCN4 peptide can be linked to the second polypeptide scaffold via a disulfide bond.

In addition to showing that the structured C-peptides produced are stabilized, the work described herein shows that a large binding interface can be grafted onto a protein scaffold in a way that preserves the biological activity of the ligand and the structure of the scaffold. This strategy is likely also effective for imparting stable secondary structure onto other biological peptide inhibitors.

Well-folded structures are often protease resistant. Therefore, one strategy for creating structured peptide inhibitors is to “graft” the critical binding residues of an unstructured peptide to a stable protein, such as to the outside of a stable protein. This must be done in such a way as to roughly preserve the three-dimensional conformation of the residues in the binding interface. This is particularly difficult for large interfaces. Previous attempts to generate high-affinity binders by grafting a larger number of residues resulted in significant destabilization of the native scaffold.

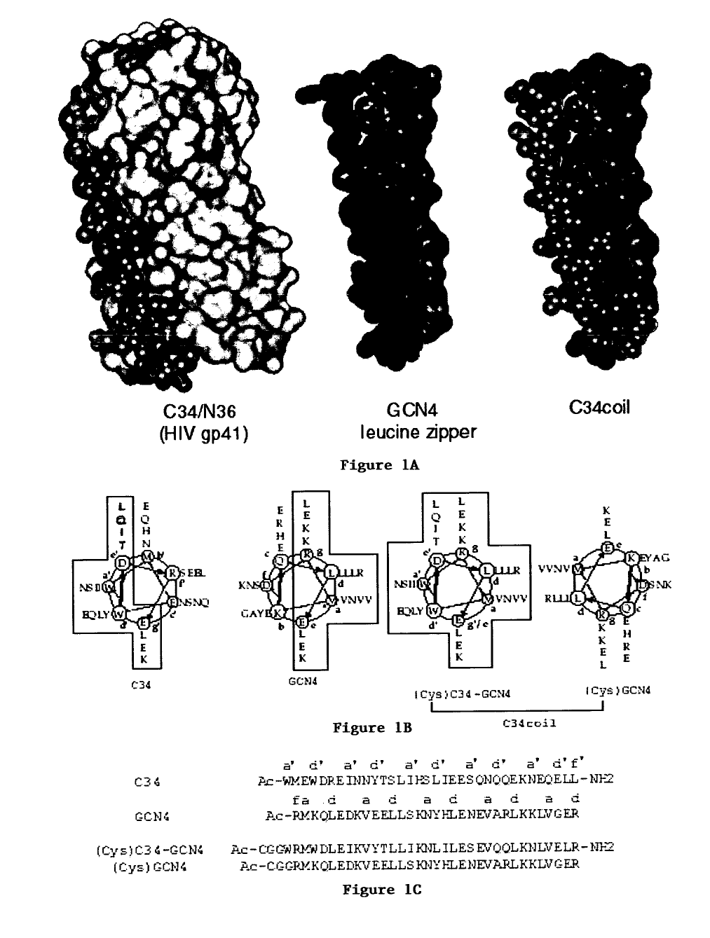

Described herein is a stable, helical, and biologically active version of C-peptide comprising the large binding epitope of C34 grafted to the GCN4 leucine zipper—a homodimeric, parallel, and stable coiled coil. For coiled coils, such as the GCN4 leucine zipper, the residues at the interface [a, d, e, and g; for a heptad in the form of (abcdefg)ₙ] are the most critical for formation of the designed structure, while those residues on the solvent-exposed face [b, c, and f] are less critical. The design described herein sought to reproduce the C34 binding interface in a helical conformation by positioning the interfacial residues of the entire C34 binding interface to the outside, solvent-exposed face of a covalently linked GCN4 leucine zipper. The resultant peptide is named “C34-GCN4 heterodimer”, “C34-GCN4 structured C-peptide” of “C34coil”. The sequence of C34-GCN4 heterodimer is represented in FIG. 1C. Also shown in FIG. 1C is the amino acid sequence of C34-GCN4 peptide which inhibits HIV-1 infection of cells (is active), but is not helical.

Applicants have shown that the C34-GCN4 heterodimer is >90% helical and a potent inhibitor of HIV-1 entry. The IC₅₀ for the C34-GCN4 heterodimer is in the nanomolar range, which is comparable to that of C34 and T-20. C34-GCN4 peptide, C34coil and their variants are therefore useful as therapeutics for HIV-1 infection. The strategy described herein can also be used for grafting stable, endogenous secondary structures to other biological peptide inhibitors.

A subject of the present invention is a structured C-peptide comprising all or a portion of a C-peptide from a virus, such as all or a portion of HIV-1 gp41 C34 peptide, and a polypeptide scaffold, such as the GCN4 leucine zipper. The components are joined or linked in structured C-peptides in such a manner that the biological activity of the C-peptide (e.g., inhibition of HIV infection of cells) is maintained and the structure of the scaffold is preserved. In particular embodiments, structured C-peptides of the present invention are stable, helical and biologically active forms of the viral C-peptide that have enhanced resistance to proteolytic degradation (relative to that of the viral C-peptide that is not linked to the polypeptide scaffold).

In one embodiment, the invention is a structured C-peptide inhibitor of HIV-1 entry into cells, such as mammalian cells, particularly human cells, in which all or a portion of C34 peptide is linked or joined to a stable protein scaffold in such a manner that the biological activity of the C34 peptide and the structure of the protein scaffold are maintained. An example of such a structured C-peptide is the C34-GCN4 peptide.

In another embodiment, a structured C-peptide of the invention comprises the complete binding epitope (19 amino acid residues) of a C-peptide (HIV-1 C34) and a protein scaffold, which is the GCN4 leucine zipper; the C-peptide, in this embodiment, is linked to (grafted onto) the outside face of the GCN4 leucine zipper, thereby producing a C34-GCN4 peptide. The C34-GCN4 peptide is linked to a second GCN4 leucine zipper, thereby producing a C34-GCN4 heterodimer or C34coil.

Helical wheel structures of C34, GCN4-pl and the C34-GCN4 heterodimer are represented in FIG. 1A. Four amino acid residues (ELEK) (SEQ ID NO: 10) overlap, as represented.

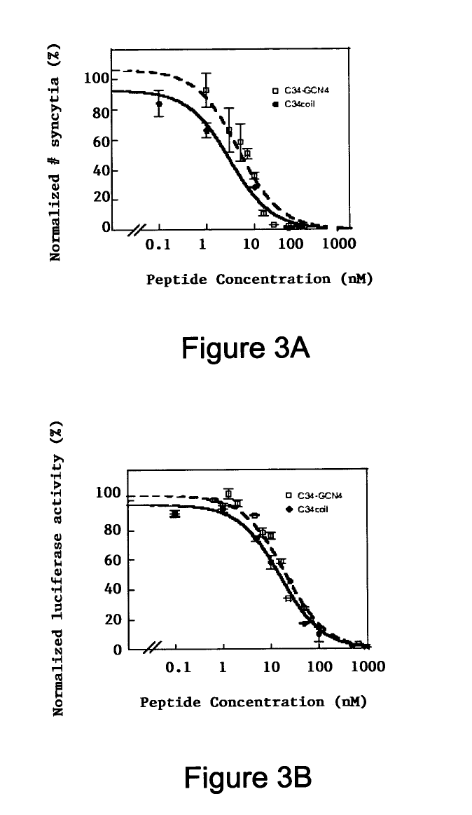

In another embodiment, a structured C-peptide comprises a modified scaffold polypeptide. The scaffold polypeptide can be modified in a variety of ways as long as the scaffold polypeptide retains the function of structuring the C-peptide in a biologically active form. Preferably, the modified scaffold polypeptide retains the function of structuring the C-peptide in a stable, helical and biologically active form. For example, in one embodiment, the present invention relates to a C34-GCN4-N16K heterodimer, in which Asn16 of both helices is changed to Lys16 (FIG. 3C).

In a further embodiment, a structured C-peptide of the present invention additionally comprises all or a portion of the HIV-1 gp41 epitope for 2F5, which is a broadly neutralizing anti-gp41 human monoclonal antibody. A C34+2F5 epitope has been shown to have the following amino acid sequence: WMEWDREINNTSLIHSLIEESQNQQEKNEQELLELDKWASLWN (SEQ ID NO: 11) (Parker C. E., et al., J. Virology, 75: 10906-10911 (2001). The amino acid sequence of C34+2F5 epitope (referred to as C34-2F5 epitope component) is shown below. It is not necessary that all of the HIV-1 gp41 epitope for 2F5 be included in the C34-2F5 epitope structured C-peptide; only a sufficient portion (one that comprises the number and appropriate composition of amino acid residues) to raise or induce production of antibodies that neutralize HIV infection. For example, the ELDKWA (SEQ ID NO: 12) core epitope previously determined for the 2F5 monoclonal antibody can be used. In one embodiment, the amino acid sequence of the C34-2F5 epitope component is WMEWDREIWWTSLIHSLIEESQNQQEKNEQELLELDKWA (SEQ ID NO: 13). In addition, any C34 or 2F5 can be mutated provided that the resulting C peptide retains function (e.g., its activity as an inhibitor of HIV-1 or its ability to cause production of or raise antibodies that neutralize HIV infection).

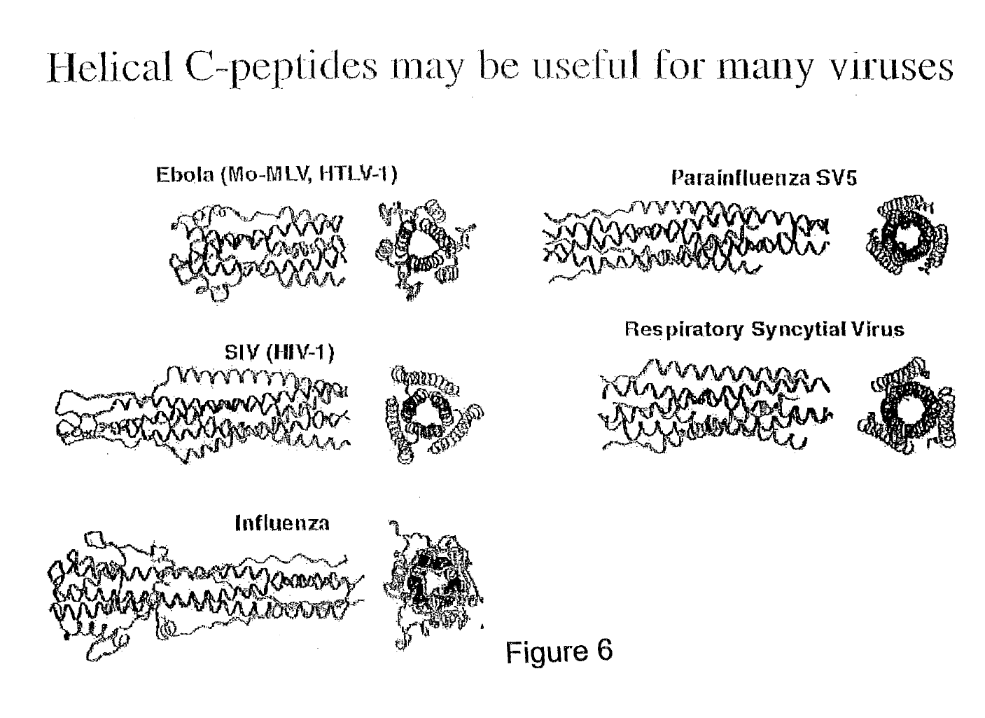

All or a portion of a selected helical C-peptide can be used in the compositions and methods of the present invention. In a particular embodiment, the C-peptide is derived from a virus. The trimer-of-hairpins motif is used for membrane fusion in many viruses, including retroviruses, orthoviruses, paramyxoviruses and filoviruses (Singh, M., et al., J. Mol. Biol., 290(5):1031-1041 (1999); Weissenhorn, W., et al., Mol. Cell., 2(5):605-616 (1998); Malashkevich, V. N., et al., Proc. Natl. Acad. Sci., USA, 96(6):2662-2667 (1999); Malashkevich, V. N., et al., Proc. Natl. Acad. Sci., USA, 98(15):8502-8506 (2001); Zhao, X., et al., Proc. Natl. Acad. Sic., USA, 97(26).14172-14177 (2000)). In some of these cases, analogous “C-peptides” have been shown to inhibit membrane fusion (Lambert, D. M., et al., Proc. Natl. Acad. Sci., USA, 93:2186-2191 (1996); Yao, Q., et al., Virology, 223:103-112 (1996); Rapaport, D., et al., EMBO J, 14(22):5524-5531 (1995)). The strategy of epitope transfer described herein can be adopted for these other viruses as well. For example, C-peptides of other viruses, such as respiratory syncytial virus, ebola virus (Mo-MLV, HTLV-1), Simian Immunodeficiency Virus (SIV), parainfluenza virus (SV5), and influenza virus, can be included in a structured C-peptide of the present invention (see FIG. 6). It is not necessary that the entire C-peptide (e.g., epitope) is used; a portion that includes an appropriate amino acid composition (number and identity of amino acid residues) such that it retains function (e.g., its activity as an inhibitor of viral function (e.g., HIV-1) or its ability to cause production of or raise antibodies that neutralize viral (e.g., HIV) infection) can be linked to a scaffold to produce a structured C-peptide. All or a portion of a selected helical C-peptide, such as all or a portion of the large binding epitope of HIV-1 gp41 C34, can be used. Finally, this strategy is likely effective for conferring stable helical structures onto other biologically active peptides.

Additionally, a variety of polypeptide scaffolds can be used in the compositions and methods of the present invention. In one embodiment, the polypeptide scaffold is the GCN4 leucine zipper.

The C-peptides and polypeptide scaffolds for use in the present invention can be obtained from natural sources (purified, partially purified), recombinantly produced, chemically synthesized or obtained from commercial sources.

The amino acid residues of the C-peptide component or the polypeptide scaffold component of the structured C-peptide can be the same as those present in the viral C-peptide or polypeptide scaffold, or can differ from the composition by the alteration, deletion, substitution, or addition of one or more (at least one) amino acid residues. The amino acid residues can be naturally occurring L- or D-amino acids, non-naturally occurring amino acids and/or modified amino acid residues. Further, the amino acid residues can be amino acid residues that occur consecutively or can be non-consecutively occurring (e.g., amino acid residues present in the viral C-peptide or polypeptide scaffold separated by additional amino acid residues, which are not included in the C-peptide component or polypeptide scaffold component; amino acid residues that are present in a different order in the C-peptide component or polypeptide scaffold component than in the viral C-peptide or polypeptide scaffold).

The present invention also relates to methods of producing structured C-peptides. Structured C-peptides of the present invention can be made using known methods of peptide synthesis, such as chemical synthetic methods (e.g., standard Fmoc chemistry). Methods for linking C-peptides onto a polypeptide scaffold are known in the art. In one approach to chemical synthesis, the C34-GCN4 heterodimer is produced through oxidation of cysteine residues, in which two separate peptides are joined to form a covalent heterodimer. In another approach, a single chain variant is produced, for example, by using an antiparallel coiled coil instead of a parallel coiled coil (as in the case of the GCN4 leucine zipper), thereby eliminating the need to oxidize two cysteine residues. Also, higher order polypeptide scaffolds (e.g., GCN4-derived oligomers, trimers and tetramers) which exhibit less supercoil than a dimer would require less conformational change of the binding epitope (C-peptide) to adopt standard helical conformation. Alternatively, structured C-peptides can be produced using recombinant methods.

The structured C-peptides of the present invention can be used in a variety of contexts. The structured C-peptides described herein inhibit (partially or totally) entry of a virus (e.g., HIV) into cells, and thus are useful prophylactically and therapeutically. Thus, the structured C-peptides of the present invention can be used in uninfected individuals (humans), and in infected individuals (e.g., to prevent or reduce infection in an uninfected individual, to reduce or prevent further infection in an infected individual). Therefore, an additional subject of the present invention is a method of inhibiting (partially or totally) viral (e.g., HIV-1) entry into cells, such as human cells, in which structured C-peptides of the present invention are administered to an individual, particularly a human, in a therapeutically effective amount. In one embodiment, the structured C-peptide is useful prophylactically as a vaccine. An example of a therapeutic use of the structured C-peptide is in the event of a needlestick injury, such as might occur in a hospital or in settings in which needles contaminated with HIV are shared. For example, an individual who is stuck with a needle and is or might be infected with HIV can receive a sufficient quantity of a structured C-peptide (therapeutically effective quantity) in one or more dose(s) in order to prevent or reduce HIV entry into cells.

The present invention also relates to a method of eliciting an immune response in an individual comprising introducing into the individual a structured C-peptide (e.g., C34-GCN4). Thus, the structured C-peptides of the present invention are vaccine candidates. One goal for a potential HIV vaccine is to elicit a neutralizing antibody against a peptide derived from the carboxy-terminal heptad repeat region of the HIV-1 gp41 envelope glycoprotein.

Structured C-peptides, such as the C34-GCN4 heterodimer described herein or a modification thereof, can be administered to an individual by a variety of routes (e.g., intravenously, orally, intramuscularly). One or more different structured C-peptides can be administered. Alternatively, blood or bone marrow can be removed from an individual infected with or thought to be infected with HIV, treated with (combined with) structured C-peptides of the present invention and returned to the individual. Structured C-peptides can also be used to reduce (partially or totally) infection of cells by HIV-1. They can be administered to an individual (particularly a human) in need of protection against infection by HIV and are useful as therapeutic peptides (to block entry of HIV into cells) and/or as vaccines (to elicit an immune response to an HIV epitope, such as the large binding epitope of C34).

In one embodiment of the present invention, the structured C-peptide is used to reduce viral, such as HIV, infection in an individual. In this embodiment, the structured C-peptide is administered, either as the structured C-peptide itself or via expression of a structured C-peptide-encoding DNA in appropriate host cells or vectors, to an individual in sufficient quantity to reduce (totally or partially) HIV infection of the individual's cells. That is, a dose of a structured C-peptide sufficient to reduce HIV infection (an effective dose) is administered in such a manner (e.g., by injection, topical administration, intravenous route) that it inhibits (totally or partially) HIV entry into cells. In one embodiment, a gene therapy approach is used to provide the effective dose, by introducing cells that express the structured C-peptide into an individual. The structured C-peptide can be administered to an individual who is HIV infected, to reduce further infection, or to an uninfected individual, to reduce infection.

More specifically, the structured C-peptides of the present invention can be administered by a variety of route(s), such as orally, nasally, intraperitoneally, intramuscularly, vaginally or rectally. In each embodiment, the structured C-peptide is provided in an appropriate carrier or pharmaceutical composition. For example, a structured C-peptide can be administered in an appropriate buffer, saline, water, gel, foam, cream or other appropriate carrier. A pharmaceutical composition comprising the structured C-peptide and, generally, an appropriate carrier and optional components, such as stabilizers, absorption or uptake enhancers, flavorings and/or emulsifying agents, can be formulated and administered in therapeutically effective dose(s) to an individual (uninfected or infected with HIV). In one embodiment, structured C-peptides are administered (or applied) as microbicidal agents and interfere with viral entry into cells. For example, a structured C-peptides can be included in a composition which is applied to or contacted with a mucosal surface, such as the vaginal, rectal or oral mucosa. The composition comprises, in addition to the structured C-peptide, a carrier or base (e.g., a cream, foam, gel, other substance sufficiently viscous to retain the drug, water, buffer) appropriate for application to a mucosal surface or to the surface of a contraceptive device (e.g., condom, cervical cap, diaphragm). The structured C-peptide can be applied to a mucosal surface, such as by application of a foam, gel, cream, water or other carrier containing the drug. Alternatively, it can be applied by means of a vaginal or rectal suppository which is a carrier or base which contains the drug or drugs and is made of a material which releases or delivers the drug (e.g., by degradation, dissolution, other means of release) under the conditions of use (e.g., vaginal or rectal temperature, pH, moisture conditions). Such compositions can also be administered orally (e.g., swallowed in capsule, pill, liquid or other form) and pass into an individual's blood stream. In all embodiments, controlled or time release (gradual release, release at a particular time after administration or insertion) of the structured C-peptide can be effected by, for example, incorporating the structured C-peptide into a composition which releases the structured C-peptide gradually or after a defined period of time. Alternatively, the structured C-peptide can be incorporated into a composition which releases the structured C-peptide immediately or soon after its administration or application (e.g., into the vagina, mouth or rectum). Combined release (e.g., release of some of the structured C-peptide immediately or soon after insertion, and over time or at a particular time after insertion) can also be effective (e.g., by producing a composition which is comprised of two or more materials: one from which release or delivery occurs immediately or soon after insertion and/or one from which release or delivery is gradual and/or one from which release occurs after a specified period). For example, a structured C-peptide can be incorporated into a sustained release composition such as that taught in U.S. Pat. No. 4,707,362. The cream, foam, gel or suppository can be one also used for birth control purposes (e.g., containing a spermicide or other contraceptive agent), although that is not necessary (e.g., it can be used solely to deliver the structured C-peptide, alone or in combination with another non-contraceptive agent, such as an antibacterial or antifungal drug or a lubricating agent). A structured C-peptide of the present invention can also be administered to an individual through the use of a contraceptive device (e.g., condom, cervical cap, diaphragm) which is coated with or has incorporated therein in a manner which permits release under conditions of use. Release of the structured C-peptide(s) can occur immediately, gradually or at a specified time, as described above. As a result, they make contact with and bind HIV and reduce or prevent viral entry into cells. In another embodiment, a structured C-peptide is administered or applied to a mucosal surface.

As used herein, a “therapeutically effective amount” is an amount of the structured C-peptide that is sufficient to inhibit (partially or totally) viral entry into cells. The quantity of structured C-peptides administered to an individual will be determined empirically, taking into consideration such factors as the age, size, extent of infection with HIV and general state of health of the recipient. The number of doses of structured C-peptides needed in a given period (daily, weekly) will also be determined empirically. Structured C-peptides can be administered alone or in combination with other drugs.

Pharmaceutical compositions which comprise the structured C-peptide (e.g., the C34-GCN4 structured C-peptide) in an appropriate carrier (e.g., a physiologically acceptable buffer) are a subject of the present invention. They are useful for preventive and therapeutic purposes and can be administered via a variety of routes.

The structured C-peptides of the present invention can also be used in a competitive assay to identify a drug that binds to the amino-terminal region of gp41. In this embodiment, the amino-terminal region of gp41 is combined with the candidate drug and the structured C-peptide and whether the candidate drug binds the amino-terminal region of gp41 is determined in the presence of the structured C-peptide. If the candidate drug binds the amino-terminal region of gp41, it is a drug that binds the amino-terminal region of gp41. For example, an amino-terminal region of gp41 is combined with a structured C-peptide of the present invention and a candidate drug to be assessed for its ability to bind the amino-terminal region of HIV gp41, thus producing a test sample, which is maintained under conditions appropriate for binding of the structured C-peptide to the amino-terminal region of gp41. A control sample, which includes the same components as the test sample, except for the candidate drug, and is handled in the same manner as the test sample, is also assessed. In both samples, binding of the reference structured C-peptide is assessed. If binding of the reference structured C-peptide occurs to a lesser extent in the presence of the candidate drug (in the test sample) than in its absence (in the control sample), the candidate drug is a drug that binds the amino-terminal region of HIV gp41. Detection of binding is assessed, for example, in a similar manner as described herein. For example, the structured C-peptide is labeled with a detectable label, such as a radiolabel or a first member of a binding pair (e.g., biotin), and the extent to which the amino-terminal region of the gp41 bears the label (after the samples have been maintained under conditions appropriate for binding of the reference structured C-peptide to the amino-terminal region of gp41) is determined. In the case in which radiolabeling is used, the extent to which the amino-terminal region of the gp41 bears the radiolabel is assessed in the test sample and compared with the extent to which the amino-terminal region of the gp41 bears the radiolabel in the control sample. If the detectable label is a first member of a binding pair (e.g. biotin), the second member of the pair (a binding partner) is added to the samples in order to detect the extent to which the amino-terminal region of the gp41 is bound by the reference structured C-peptide. This can be done directly or indirectly (e.g., by adding a molecule, such as an antibody or other moiety which binds the second member of the binding pair). Less of the label will be present on the amino-terminal region of the gp41 if the candidate drug has inhibited (totally or partially) binding of the structured C-peptide to the amino-terminal region of the gp41. If binding occurs to a lesser extent in the test sample (in the presence of the candidate drug) than in the control sample (in the absence of the candidate drug), then the candidate drug is a drug that binds the amino-terminal region of HIV gp41.

The present invention is illustrated by the following examples, which are not intended to be limiting in any way.

### EXAMPLES

Material and Methods

Peptide Synthesis and Purification

Peptides were synthesized by standard Fmoc chemistry with acetylated N-termini and either free or amidated C-termini (sequences shown in FIG. 1C). The identities of all peptides were confirmed by matrix-assisted laser desorption ionization mass spectroscopy (MALDI-MS) (Perceptive Biosystems Voyager Elite) to be within 0.1% of the predicted masses. Reverse-phase high-performance liquid chromatography (HPLC) (Vydac C 18 column) was used to purify the peptides. To synthesize the heterodimer, approximately equimolar amounts of the (Cys)C34-GCN4 and (Cys)GCN4 peptides were mixed in 6 M guanidine hydrochloride and 100 mM Tris, pH 8.0. After 24 hours of air oxidation, the heterodimer was purified by reverse-phase HPLC. The purity and molecular weight of the heterodimer were verified by liquid chromatography-mass spectroscopy (LC MS) (Finnegan-mat LCQ). Peptide concentrations were determined by absorbance at 280 nm in 6 M guanidine hydrochloride, using extinction coefficients of 5690 M⁻¹cm⁻¹ for tryptophan, 1280 M⁻¹cm⁻¹ for tyrosine, and 120 M⁻¹cm⁻¹ for cystine (H. Edelhoch, Biochemistry 6, 1948-1954 (1967)).

Circular Dichroism (CD) Spectroscopy and Urea Denaturation

CD spectra were measured on an Aviv 60DS spectrapolarimeter at 25° C. in phosphate buffered saline (PBS; 10 mM sodium phosphate, 150 mM NaCl, pH 7.0). The peptide concentrations used were: 20 μM C34, 16 μM C34-GCN4 and 1 μM C34coil. For chemical denaturation experiments, the [Θ]₂₂₂ values of C34coil (1 μM) were measured as a function of guanidine hydrochloride concentration in PBS. Guanidine concentrations were measured by refractometry. The titrations were fitted to the standard six-parameter two-state transition equation using weighted averages to yield standard folding free energy (C. N.Pace, Methods Enzymol. 131, 266-280 (1986)).

Gel Filtration

Gel filtration chromatography was performed on a TosoHaas G4000SWXL column at room temperature, using PBS as the running buffer. C34coil (25 μL and approximately 100 μM) was injected into the column running at 1 mL/min. A Bio-Rad gel filtration standard was run as a molecular weight standard.

Proteoloysis

Proteinase K (Sigma) was added to the peptides (at concentrations of either 10 μM or 20 μM) in PBS and incubated at 37 C. For the indicated relative ratios of proteinase K digestion, the amounts of proteinase K and incubation times were: 0.002 μg/mL and 1 minute for 1×, 0.02 μg/mL and 1 minute for 10×, 0.2 μg/mL and 60 minutes for 600×, and 0.2 μg/mL and 17 hours for 1000×. After incubation, the reactions were quenched by bringing the solutions to a final concentration of 1 mM phenylmethanesulfonyl fluoride (PMSF) and 5% acetic acid. The reaction products were run on a Microsorb-MV C18 column at a speed of 1 mL/min and a gradient of 1% acetonitrile/minute.

Viral Infectivity and Syncytia Assay

Assessment of viral infectivity and syncytia formation were carried out as described by Eckert et al., Cell, 99:103-115 (1999). In both inhibition assays, peptide stocks were dissolved in dimethylsulfoxide and their concentrations determined by absorbance in guanidine at 280 nm. The final concentration of dimethylsulfoxide in tissue culture media was 1% in all experiments. Inhibition of cell-cell fusion (syncytia formation) (G. J. Nabel, Nature 410, 1002-1007 (2001); D. C. Chan, C. T. Chutkowski, P. S. Kim, Proc. Natl. Acad. Sci. 95, 15613-15617 (1998)) was assayed by co-culturing 3×10⁴ Chinese hamster ovary (CHO) cells expressing HXB2 envelope and tat and rev with 5×10⁴ HeLa cells expressing CD4 and LTR-driven β-galactosidase in the presence of varying concentrations of peptides. The CHO and HeLa cells were incubated in the presence of peptides for 20 hours, and stained with 5-bromo-4-chloro-3-indolyl-β-D-galactoside (X-gal) to detect the syncytia, which were visualized by microscopy and counted manually. In the viral infectivity assay, the ability of the peptides to inhibit HIV-1 infection was assayed using a recombinant luciferase-encoding HIV-1 (G. J. Nabel, Nature 410, 1002-1007 (2001); D. C. Chan, C. T. Chutkowski, P. S. Kim, Proc. Natl. Acad. Sci. 95, 15613-15617 (1998)). 293T cells were co-transfected with an envelope-deficient HIV-1 genome with a luciferase gene (NL43LucR-E-) [B. K. Chen, K. Saksela, R. Andino, D. Baltimore, J. Virol. 68, 654-660 (1994)] and the HXB2 gp160 expression vector (pCMVHXB2 gpl60). Low-speed centrifugation was used to clear the viral supernatants of cellular debris. The supernatant containing the viruses was used to infect HOS cells expressing CD4 and CXCR4 (HOS-CD4/Fusin cells, N. Landau, National Institutes of Health AIDS Reagent Program) in the presence of varying concentrations of peptides. The HOS cells were harvested 48 hours post-infection, and luciferase activity was monitored using a Wallac AutoLumat LB953 luminometer. In both assays, the IC₅₀, the concentration needed to inhibit 50% of cell-cell fusion events or luciferase activity, was calculated from fitting the data to the equation, y=k/(1+[peptide]/IC₅₀), where y=number of syncytia or luciferase activity and k is a scaling constant.

Results

Protein grafting, the transfer of a binding epitope of one ligand onto the surface of another protein, is a powerful technique for presenting peptides in pre-formed and active three-dimensional conformations. Its utility, however, has been limited by low biological activity of the designed ligands and low tolerance of the protein scaffolds to surface substitutions. As described herein, the complete binding epitope (19 nonconsecutive amino acids) of a HIV-1 C-peptide, which is derived from the carboxy-terminal region of HIV-1 gp41 and exhibits potent antiviral activity in humans, was grafted onto the surface of a GCN4 leucine zipper. The designed peptide, named C34coil, displays a potent antiviral activity approaching that of the native ligand. Moreover, while the linear C-peptide is unstructured and sensitive to degradation by proteases, C34coil is well-structured, conformationally stable, and exhibits increased resistance to proteolytic degradation compared to the linear peptide. In addition to being a structured antiviral inhibitor, C34coil serves as the basis for the development of a new class of immunogens which presents the C-peptide region of gp41 in a structured conformation. The data described herein demonstrate that with the appropriate scaffold protein, protein grafting can be used to create ligands with potent biological activity and novel structural properties.

Specifically, described herein, is a helical, stable and biologically potent C-peptide which was constructed by grafting all 19 amino acids of the binding epitope of C34 onto the surface of a GCN4 leucine zipper, a stable, homodimeric coiled coil (E. K. O'Shea, J. D. Klemm, P. S. Kim, T. Alber, Science 254, 539-544 (1991) (FIG. 1A). The coiled coil is a good candidate to act as a scaffold protein, as it is a stable and protease-resistant structure (M. Lu, S. C. Blacklow, P. S. Kim, Nat. Struct. Biol. 2, 1075-1082 (1995); A. Lupas, Trends Biochem. Sci. 21, 375-382 (1996); P. A. Bullough, F. M. Hughson, J. J. Skehel, D. C. Wiley, Nature 371, 37-43 (1994)), and helix bundles can effectively present binding epitopes on their surfaces (H. Domingues, D. Cregut, W. Sebald, H. Oschkinat, L. Serrano, Nat. Struct. Biol. 6, 652-656 (1999); N.J. Zondlo, A. Schepartz, J. Am. Chem. Soc. 121, 6938-6939 (1999); G. Ghirlanda, J. D. Lear, A. Lombardi, W. F. DeGrado, J. Mol. Biol. 281, 379-391 (1998)). The GCN4 leucine zipper is a particularly attractive scaffold protein for C34, as it contains four full heptad repeats which matches the length of C34, and its moderate stability allows for conformational flexibility in the grafted residues. To design the sequence of the helical C-peptide, it was found that in coiled coils, the residues forming the hydrophobic core are the most critical for formation of the helical structure, while the solvent-exposed residues are less critical for stability or dimerization (A. Lupas, Trends Biochem. Sci. 21, 375-382 (1996); E. K. O'Shea, R. Rutkowski, P. S. Kim, Cell 68, 699-709 (1992) 29, 32). Therefore, as a first step, all 19 amino acids were used at positions a, d, e and g of GCN4 (for an amino acid sequence with a heptad repeat of the form (abcdefg)ₙ), which comprise the complete hydrophobic core (FIGS. 1B and 1C). In addition, all 19 residues at the a′, d′, e′ and g′ positions of C34 (prime denotes the heptad positions of C34), which constitute the complete epitope for binding to the N-peptide region of gp41 (D. C. Chan, D. Fass, J. M. Berger, P. S. Kim, Cell 89, 263-273 (1997); W. Weissenhorn, A. Dessen, S. C. Harrison, J. J. Skehel, D. C. Wiley, Nature 387, 426-430 (1997); K. Tan, J. Liu, J. Wang, S. Shen, M. Lu, Proc. Natl. Acad. Sci. 94, 12303-12308 (1997)), were transferred onto the solvent-exposed f, b, c and e positions, respectively, of the GCN4 peptide (FIGS. 1B and 1C). The four amino acids at position g′ of C34 are identical with those at position e of GCN4 (FIGS. 1B and 1C), which allowed both the complete hydrophobic core of GCN4 and the complete binding epitope of C34 to be recapitulated in the designed peptide. The designed peptide, denoted C34-GCN4, is 34 residues in length: 19 from C34, 19 from GCN4, with 4 overlapping residues.

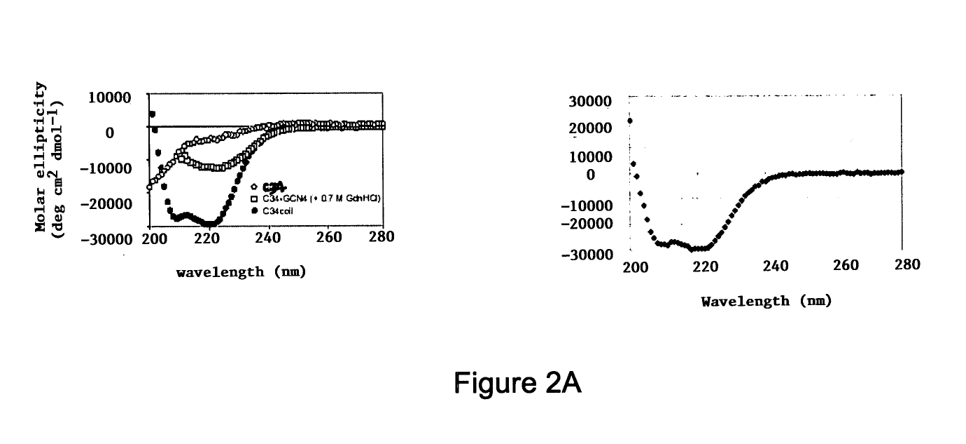

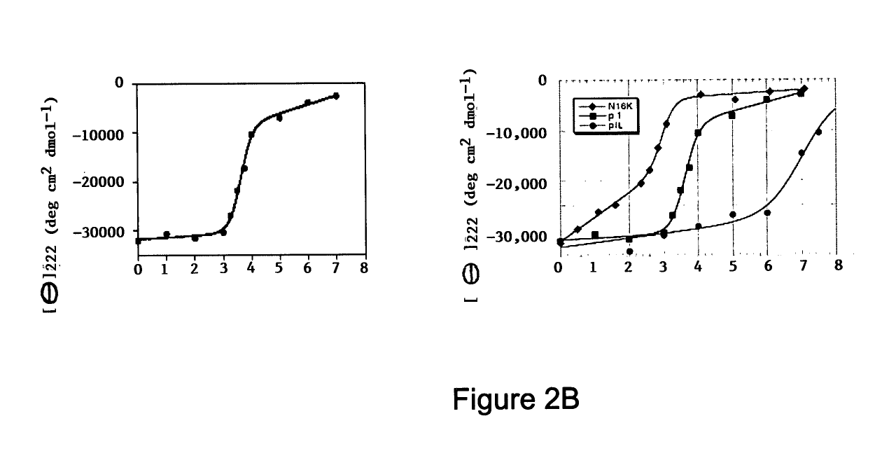

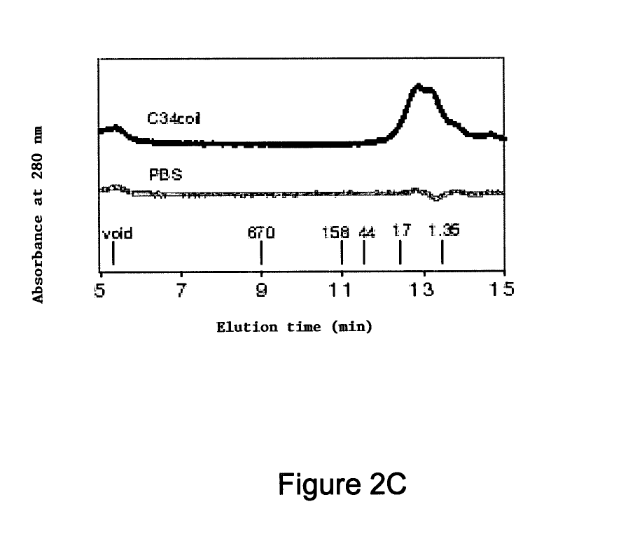

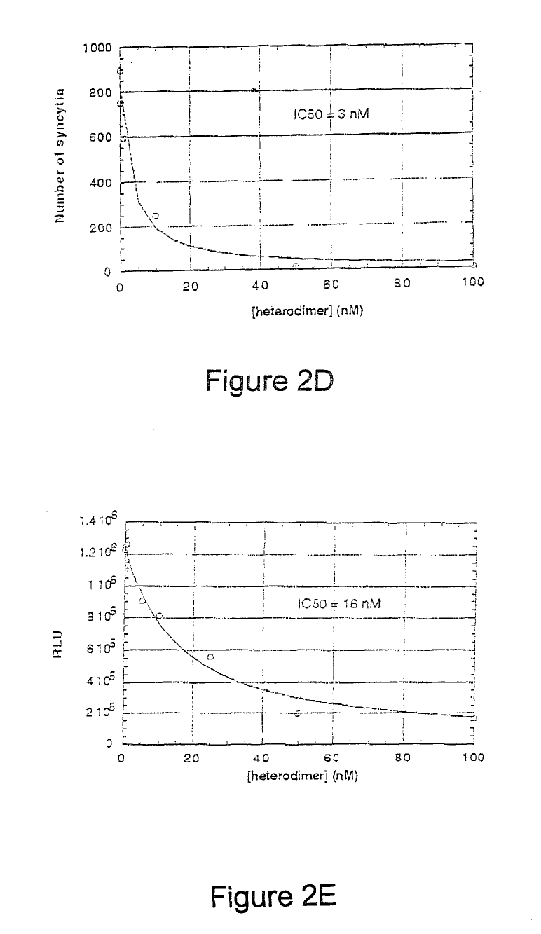

The C34-GCN4 peptide is very hydrophobic (FIG. 1C), and as a result, exhibited low solubility (less than 1 μM in PBS). The circular dichroism (CD) spectrum of the C34-GCN4 peptide (in a low amount of guanidine hydrochloride (about 0.7 M) to increase peptide solubility) indicated a mostly random coil structure with a [Θ]₂₂₂ value of −12,000 deg cm² dmol⁻¹ (FIG. 2A). Although this value is slightly higher than that of C34 (Lu, et al., J. Biomol. Struct. Dyn., 15(3):465-471 (1997)), the data indicate that under the experimental conditions, the C34-GCN4 peptide is unable to form a stable, helical coiled coil structure.

To overcome the poor helical content and solubility of the C34-GCN4 peptide, a disulfide-linked heterodimer, denoted C34coil (alternatively referred to as the C34-GCN4 heterodimer), composed of two peptides: (Cys)C34-GCN4 and (Cys)GCN4, was made (FIG. 1B). An N-terminal Cys-Gly-Gly linker was added to both peptides and air oxidation linked the two Cys residues. The resultant heterodimer was purified from the two homodimers by reverse phase HPLC. Compared to the C34-GCN4 peptide, C34coil is more soluble since the second helix of GCN4 contains many hydrophilic amino acids, and more helical since the disulfide-linkage stabilizes the helical conformation of coiled-coil dimers (E. K. O'Shea et al., Science, 243:538-542 (1989)). Indeed, C34coil exhibited enhanced solubility (up to 100 μM in PBS) and a helical content of over 90% of the expected value (FIG. 2A) despite substantial mutations to the surface of the GCN4 leucine zipper.

To test the effect of protein stability on the biophysical properties and inhibitory potency of the heterodimer, two variants of the C34 coil with different amino acids in the hydrophobic core were synthesized. One is the C34-GCN4-N16K heterodimer, in which Asn 16 of both helices is changed to Lys 16 (FIG. 3C). This heterodimer is expected to be less stable because the N16K mutation in GCN4 decreases the melting temperature by about 10° C. (Gonzalez, et al., Nature Struct. Biol., 3:1011-1018 (1996)). Another variant is the C34-GCN4-pIL heterodimer, in which the four residues at position a are changed from Val or Asn to Ile. This heterodimer is expected to be very stable, as the same mutations in GCN4 increases the melting temperature to greater than 100° C. (Harbury, et al., Science, 262:1401-1407 (1993)). In both cases, the GCN4 variants were shown to behave as clean dimers (Gonzalez, et al., Nature Struct. Biol., 3:1011-1018 (1996); Harbury, et al., Science, 262:1401-1407 (1993)).

The disulfide linkage has been observed to markedly stabilize a coiled coil dimer. For example, the disulfide-linked GCN4 homodimer exhibits a transition midpoint of over 100° C. under temperature denaturation, compared to 56° C. for the noncovalent GCN4 homodimer (at a temperature of 10 μM (Gonzalez, et al., Nature Struct. Biol., 3.1011-1018 (1996)). The stability of the C34 coil was assessed by the chemical denaturation using guanidine hydrochloride. The C34 coil unfolds with a midpoint concentration (C_{M}) of 3.6 M guanidine, with a free energy of folding (ΔG_{fold}) of −9.3 kcal/mol (FIG. 2B). It is therefore slightly more stable than the C34-GCN4-N16K heterodimer, which unfolds with a C_{M} of 3.0 M guanidine and ΔG_{fold} of −9.0 kcal/mol. The lower stability of the C34-GCN4-N16K heterodimer agrees with the observation that a GCN4-N 16K homodimer is slightly less stable than a wildtype GCN4 homodimer (Gonzalez, et al., Nature Struct. Biol., 3:1011-1018 (1996)). Meanwhile, the C34-GCN4-pIL heterodimer is very stable, unfolding with a C_{M} of 7.0 M guanidine (an accurate fit for ΔG_{fold} could not be obtained because of a poor baseline for the unfolded state). This is consistent with the observation that the GCN4-pIL homodimer is thermally extremely stable (Harbury, et al., Science, 262:1401-1407 (1993)).

The helical structure of C34coil is stable, as shown by chemical denaturation (FIG. 2B). Importantly, C34coil is monomeric and exhibits no aggregation under physiological conditions, as determined by gel filtration chromatography (FIG. 2C). Thus, C34coil exhibits a stable, helical structure despite substantial mutations to the surface of the GCN4 leucine zipper, and the pre-formed helical conformation of the C34 binding epitope does not induce hydrophobic aggregation.

It is possible that in C34coil, the critical binding residues of C34 are repositioned in such a way as to render them biologically inactive. To test how well the HIV-1-neutralizing epitope was recapitulated, two assays were used to measure the potency of the designed peptides in inhibiting HIV-1 envelope-mediated membrane fusion. The biological activity of the designed peptides were first measured by their ability to inhibit fusion of HeLa cells expressing CD4 and chemokine receptors and CHO expressing HIV envelope protein (D. C. Chan, C. T. Chutkowski, P. S. Kim, Proc. Natl. Acad. Sci. 95: 15613-15617 (1998)).

In both the cell-cell fusion (FIG. 3A) and viral infectivity (FIG. 3B) assays, C34coil exhibits potent antiviral activities, with IC₅₀ values of 3.1±0.8 nM and 16±2 nM, respectively, which are within an order of magnitude of those of C34 (D. C. Chan, C. T. Chutkowski, P. S. Kim, Proc. Natl. Acad. Sci. 95: 15613-15617 (1998)). In addition, the GCN4 homodimer, a covalent homodimer of (Cys)GCN4 (FIG. 1C), shows no activity in both assays (FIGS. 3A and 3B), confirming that the C34 binding epitope, and not the GCN4 portion, is responsible for the inhibitory activity of C34coil. The potent inhibitory activity of C34coil demonstrates that the helical C34 epitope is presented in a biologically active conformation The potent antiviral activity of C34coil suggests that residues at the solvent-exposed face of the C-peptide helix (opposite from the face that binds to gp41, at b, c and f positions of the heptad repeat) are not crucial for activity. Consistent with this observation, the C34-GCN4 peptide, which contains all the key binding residues of C34 but features completely different residues at the solvent-exposed positions, shows strong inhibitory potency (FIGS. 3A and 3B). The remarkable tolerance to substitutions indicates that significant amino acid substitutions (for helix stabilization, for example) can be used to create an even more potent linear C-peptide than C34 or T-20.

As shown above, C34coil inhibits cell-cell fusion. This inhibitory potency is within an order of magnitude of C34 (D. C. Chan, C. T. Chutkowski, P. S. Kim, Proc. Natl. Acad. Sci. 95: 15613-15617 (1998)). Interestingly, the C34 coil, although only weakly helical, also inhibits cell-cell fusion potently. This behavior is similar to that of C34 (Wild, C. T., et al., Proc. Natl. Acad. Sci., USA, 95:9770-9774 (1994); Lu, M. et al., J. Biomol. Struct. Dyn., 15(3):465-471 (1997)). For the two heterodimer variants, the C34-GCN4-N16K potently inhibits cell-cell fusion, whereas the C34-GCN4-pIL shows no measurable inhibitory activity up to 1 μM.

In a second assay, the ability of the peptides to inhibit HIV infection of HOS cells expressing CD4 and CXCR4 was measured, using a luciferase-based reporter assay (D. C. Chan, C. T. Chutkowski, P. S. Kim, Proc. Natl. Acad. Sci. 95: 15613-15617 (1998); Chen, B. K., et al.,J. Virol., 68(2):645-660 (1994)). The C34-GCN4 peptide, the C34 coil and the C34-GCN4-N16K heterodimer exhibit potent antiviral activity. The C34-GCN4-pIL heterodimer again shows no measurable activity up to 1 μM.

Table Inhibitory activities of various peptides in the cell-cell fusion assay and viral infectivity assay. C34 data is taken from D. C. Chan, C. T. Chutkowski, P. S. Kim, Proc. Natl. Acad. Sci. 95: 15613-15617 (1998). The GCN4 homodimer is a covalent homodimer of CGG-GCN4.

| | Cell-Cell Fusion Assay | Viral Infectivity Assay |
|---|---|---|
| Peptide | (IC50 in nM) | (IC50 in nM) |
| C34-GCN4 | 5.0 | 16 |
| C34 coil | 3.0 | 16 |
| C34-GCN4-N16K | 1.5 | 10 |
| heterodimer | | |
| pC34-GCN4-pIL | none | none |
| heterodimer | | |
| C34 | 0.6 | 2.1 |
| GCN4 homodimer | none | none |

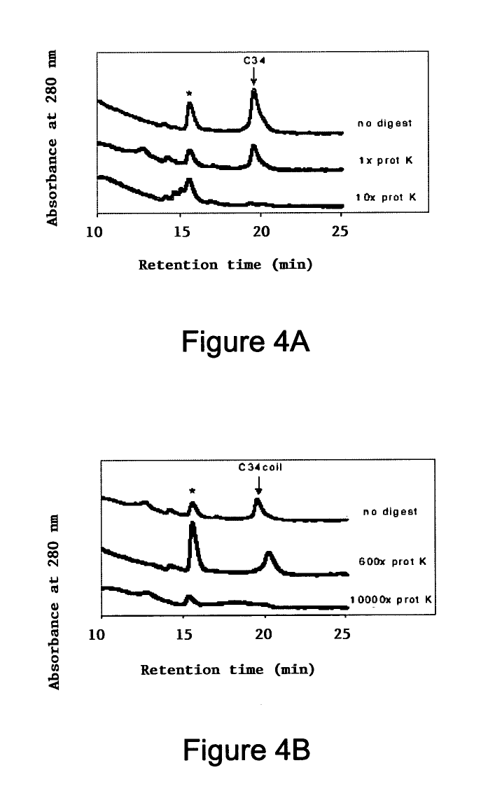

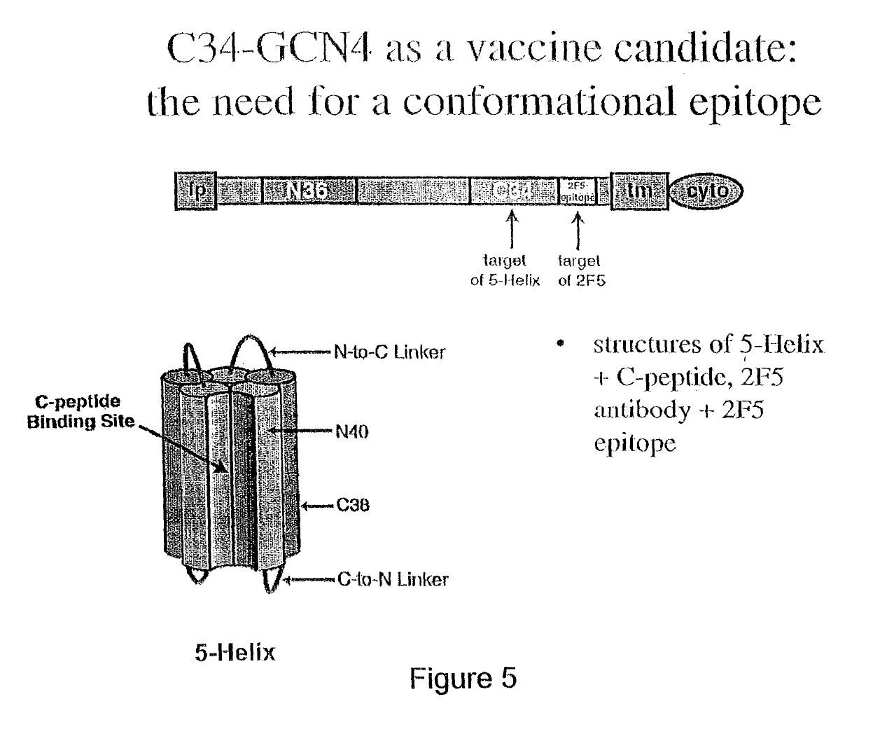

The stable, helical structure of C34coil is expected to give rise to an increased resistance to proteolytic degradation compared with linear peptides, as proteases are most active on unfolded substrates. The sensitivities to proteolytic degradation of C34coil and the linear peptide C34 by digestion with proteinase K, a protease of broad substrate specificity, were compared and the reaction products were analyzed by reverse-phase HPLC. C34coil is approximately 1000-fold more resistant to degradation by proteinase K than C34 (FIGS. 4A-4B). Because of its increased resistance to proteolytic degradation and potent inhibitory activity, it is likely that the C34coil or its variants are useful therapeutics for HIV-1 infection in addition to the C-peptides being currently tested (J. M. Kilby et al., Nat. Med. 4, 1302-1307 (1998)).

It is also likely that the C34coil is an effective immunogen in eliciting antibodies which recognize the C-peptide region of gp41 in a helical conformation and block membrane fusion (M. J. Root, M. S. Kay, P. S. Kim, Science 291, 884-888 (2001)). Moreover, against C34coil, the antibodies would recognize only the highly conserved binding epitope of C34, compared to linear epitopes containing both conserved and non-conserved residues. C34coil could also be fused to a 2F5 epitope constrained into a β-turn (whose conformation is important for recognition by the 2F5 antibody (E. F. Pai, M. H. Klein, A. Pedyczak, WO 00/61618; J. Ho, K. S. MacDonald, B. H. Barber, Vaccine 20, 1169-1180 (2002)) to form an extended conformationally stabilized epitope.

The potent biological activity of C34coil likely stems in part from the grafting of a large number of key binding residues in the C34 binding epitope, as interactions along the entire interface between the C-peptides and N-peptides are important for the stability of the six-helix bundle (M. Lu et al., J. Virol. 75, 11146-11156 (2001)). The data described herein demonstrate that the coiled coil is a structurally stable scaffold for a large number of surface substitutions as long as the entire hydrophobic core of the coiled coil is preserved (which was not the case in another study (H. Domingues, D. Cregut, W. Sebald, H. Oschkinat, L. Serrano, Nat. Struct. Biol. 6, 652-656 (1999)), and is consistent with previous studies which showed the native fold of a protein can tolerate large-scale substitutions if the substitutions are appropriately chosen (S. Dalal, S. Balasubramanian, L. Regan, Nat. Struct. Biol. 4, 548-552 (1997)). Despite presenting the full binding epitope in a helical conformation, however, C34coil is a slightly less potent inhibitor than the linear peptide C34. The entropic gain in binding energy from a pre-formed helical scaffold may be offset by the energy required to change the C34 binding epitope from a coiled-coil conformation (E. K. O'Shea, J. D. Klemm, P. S. Kim, T. Alber, Science 254, 539-544 (1991); A. Lupas, Trends Biochem. Sci. 21, 375-382 (1996)) to the active helical conformation (D. C. Chan, D. Fass, J. M. Berger, P. S. Kim, Cell 89, 263-273 (1997); W. Weissenhorn, A. Dessen, S.C. Harrison, J. J. Skehel, D. C. Wiley, Nature 387, 426-430 (1997); K. Tan, J. Liu, J. Wang, S. Shen, M. Lu, Proc. Natl. Acad. Sci. 94, 12303-12308 (1997)). Overall, the GCN4 coiled coil is a useful protein scaffold for large-scale surface substitutions, as it is sufficiently stable to retain its helical conformation, but not overly stable as to lock the binding epitope into an inactive conformation.

The presentation of protein sequences in pre-formed, active three-dimensional conformations is a powerful strategy for improving their biomedical properties. For example, linear peptides and proteins that are attractive candidates for therapeutics and immunogens often exhibit three-dimensional conformations which limit their practical utility. As shown herein, with an appropriate scaffold protein, protein grafting of a large binding epitope can be performed in a way that exhibits both a potent biological activity and a novel three-dimensional structure. The utility of the protein grafting strategy can be augmented by combinatorial selection to optimize the potency or stability of the designed peptide (J. W. Chin, A. Schepartz, J. Am. Chem. Soc. 123, 2929-2930 (2001). In addition, a single-chain variant based on an antiparallel coiled coil can be engineered, which would allow for recombinant expression and eliminate the need for cysteine oxidation to link together two separate peptides. Also, compared to the GCN4 used in the current study, coiled coils with lower stability would allow the anti-HIV-1 epitope to more closely adopt its native conformation when bound to gp41, resulting in peptides of even higher antiviral potency.

Discussion

Therapeutic. Many C-peptides have been shown to inhibit HIV entry into cells in cell culture (Wild, C. T., et al., Proc. Natl. Acad. Sci, USA, 91:9770-9774 (1994); Chan, D., et al., Proc. Natl. Acad. Sci, USA, 95:15613-15617 (1998)). One C-peptide, T-20, is effective in reducing plasma viral load in humans (Kilby, J. M., et al., Nat. Med., 4(11).1302-1307 (1998)). However, large amounts of T-20 are needed to achieve significant efficacy. This may be due to its unstructured conformation in the absence of gp41, which makes it sensitive to proteolytic degradation. The construction of a structured C-peptide which contains the complete binding epitope of C34 is described herein. The C34-GCN4 heterodimer, also referred to as the C34coil, is highly helical in the absence of gp41, with good conformational stability as measured by chemical denaturation. It is also a potent inhibitor of HIV-1 entry with IC₅₀ values in the nanomolar range, within an order of magnitude of C34. The C34-GCN4 heterodimer or its variants are useful therapeutics for HIV-1 infection, as its structured conformation would be more resistant to proteolytic degradation than unstructured C-peptides.

Vaccine candidate. A successful HIV vaccine will likely need to elicit both cell-mediated (Barouch, D. H., et al., Science, 290:486-492 (2000); Burton, D., et al., Nat. Med., 4(5):495-498 (1998)) and humoral immunity (Baba, T., et al., Nat. med., 6(2):200-206 (2000); Mascola, J. R., et al., Nat. Med., 6(2).207-210 (200)). To achieve humoral immunity, it is necessary to target the only viral protein on the virion surface, the HIV envelope protein. For instance, the three antibodies currently known to neutralize both laboratory and primary HIV isolates all target the HIV envelope protein (F105 and 2G12 target gp120, 2F5 targets gp41) (Baba, T., et al., Nat. med., 6(2):200-206 (2000); Muster, T., et al., J. Virol., 67(11):6642-6647 (1993)). Moreover, passive transfer of broadly neutralizing antibodies to macaque monkeys conferred protection against infection by simian-human immunodeficiency virus (a variant of simian immunodeficiency virus which expresses HIV envelope (Baba, T., et al., Nat. med., 6(2):200-206 (2000); Mascola, J. R., et al., Nat. Med., 6(2):207-210 (200)). Eliciting a neutralizing response in the host using the viral envelope protein is difficult because few epitopes on the envelope protein are accessible (Wyatt, R., et al., Science, 280:1884-1888 (1998); Wyatt, R., et al., Nature, 393(6686):705-711 (1998)), and immunogens have failed to represent the native conformation of the protein (Burton, D., et al., Nat. Med., 4(5):495-498 (1998)).

Two lines of evidence indicate that the C-peptide region of gp41 as displayed on the virion surface is accessible to circulating proteins, and that binding to the C-peptide region inhibits HIV entry. First, a designated protein called “5-Helix” binds to the C-peptide region with high affinity, and potently inhibits the infection by several diverse strains of HIV (Root, M., et al., Science, 291:884-888 (2001)). Second, the broadly neutralizing antibody 2F5 targets a region of gp41 just C-terminal to the C-peptide (Purtscher, M., et al., AIDS Res. Human Retroviruses, 10(12):1651-1658 (1994); Muster, T., et al., J. Virol., 67(11):6642-6647 (1993); Conley, A. J., et al., Proc. Natl. Acad. Sci., USA:91:3348-3352 (1994); Purtscher, M., et al., AIDS, 10:587-593 (1996)). A recent study indicates that C34 may overlap with the 2F5 epitope by 6 residues (Parker, C. E., et al., J. Virol., 75(22):10906-10911 (2001)), indicating that at least part of the C-peptide region is an accessible target for antibody binding. The evidence that two broadly neutralizing agents can bind to the C-peptide region and inhibit HIV entry presents a compelling case that peptides derived from the C-peptide/2F5 epitope regions are attractive vaccine candidates.

However, immunization with linear C-peptides or peptides containing the 2F5 epitope have thus far failed to elicit neutralizing antibodies. For example, immunization with C34 or T-20 elicits a strong immune response even in the absence of a carrier protein, but the antibodies fail to recognize the envelope protein with our without incubation of soluble CD4 (to mimic receptor binding), and the antibodies are not neutralizing (Rosny, E., et al., J. Virol., 75(18):8859-8863 (2001)). Similarly, immunization with linear peptides containing the 2F5 epitope elicit an adequate immune response, but the antibodies fail to neutralize HIV infection (Muster, T., et al., J. Virol., 68(6):4031-4034 (1994); Eckhart, L., et al., J. Gen. Virol., 77:2001-2008 (1996)). This indicates that, like other vaccine candidates derived from the HIV envelope glycoprotein (including gp120), the C-peptide region/2F5 epitope has not been presented in a conformation that can elicit neutralizing antibodies (Burton, D., et al., Nat. Med., 4(5):495-498 (1998)). Since 5-Helix binds to the C-peptide region as a helix (Root, M. J., et al., Science, 291:884-888 (2001)), the C34-GCN4 heterodimer and its variants —as structured, helical C-peptide—serve as the basis for a new class of vaccine candidates. Also, since the 2F5 antibody binds to its epitope as a β-turn (Pai, E. F., et al., WO 00/61618), the C34-GCN4 heterodimer can be fused to a 2F5 epitope constrained into a β-turn (using known β-turn constraining methods (Cochran, A. G., et al., J. Am. Chem. Sco., 123(4):625-632 (2001)) to form conformationally stabilized epitopes which may elicit neutralizing antibodies and serve as potential vaccine candidates.

Comparison of heterodimer mutants. Successful replication of the C34 binding epitope requires a suitable helical protein scaffold. While the importance of stability in choosing a protein scaffold has been well recognized (Nygren, P., et al., Curr. Opin. Struct. Biol., 7:463-469 (1997); Femandez-Carneado, J., et al., Biopolymers, 55:451-458 (2001)), the role of conformational flexibility is less studied. To reproduce the C34 binding epitope, the protein scaffold must be sufficiently stable to retain its helical conformation in the presence of 15 surface mutations, but not so stable as to lock the C34 binding epitope into an inactive helical conformation. For example, the superposition of the backbones of a C34 helix and a GCN4 helix shows good but not perfect agreement (root mean squared deviation of about 1 Å), with the differences mostly due to the absence of the leucine zipper supercoil in the C34 helix. For the C34-GCN4 heterodimer to be biologically active, its hydrophobic core must be sufficiently labile to allow the C34 binding epitope on the surface to change from a coiled coil to the active helical conformation. Note that although the active conformation of the binding epitope in the C34-GCN4 heterodimer is most likely to resemble the conformation of C34 in the crystal structure of the six-helix bundle, it is also possible that the binding of the stable C34-GCN4 heterodimer induces a structural change in the N-peptide coiled coil instead.

As described herein, two variants of the C34-GCN4 heterodimer of varying stabilities due to mutations in the hydrophobic core were synthesized and analyzed (FIG. 3C). Interestingly, the less stable C34-GCN4-N16K heterodimer showed slightly greater inhibitory potency in both biological assays compared to the C34-GCN4 heterodimer. Meanwhile, the C34-GCN4-pIL heterodimer showed no measurable inhibition up to 1 μM. The lack of inhibitory potency of the C34-GCN4-pIL heterodimer is likely partially due to the low solubility of this peptide. Furthermore, it also indicates that the hydrophobic core of the C34-GCN4-pIL heterodimer is too rigid to allow the C34 epitope to adopt the active conformation.

Helix stabilization strategies of short peptides aim to increase their helical content and helix propensity, and ultimately improve their biological activity (Andrews M. J. I., et al., Tetrahedron, 55:11711-11743 (1999), Chapter 2). However, the C34-GCN4 heterodimer (and the C34-GCN4-N16K heterodimer), despite successfully presenting the C34 binding epitope in a helical conformation, is not a more potent inhibitor than C34. For the C34-GCN4 heterodimer, the entropic stabilization of the helical scaffold is likely partially offset by the energy required to change the C34 binding epitope from a coiled coil conformation (O'Shea, E. K., et al., Cell, 68:699-709 (1992)) to the active helical conformation (Chan, D., et al., Cell, 89:263-273 (1997); Weissenhom, W., et al., Nature, 387:426-430 (1997)), similar to but not as dramatic as what may be observed for the C34-GCN4-pIL heterodimer.

Interestingly, the C34-GCN4 peptide itself showed remarkably strong inhibitory potency. The lack of observed helical structure (at 1 μM peptide concentration) suggests that the peptide failed to form stable, oligomeric coiled coil structures, and that the C34-GCN4 peptide may be inhibiting membrane fusion as a weakened C34 peptide. Even with 40% mutations compared to C34, its inhibitory potency is remarkably strong (within an order of magnitude for both the cell-cell fusion and viral infectivity assays, Table). This indicates that the solvent-exposed face of the C-peptide (the b, c and f residues) is remarkably tolerant to changes, indicating that various helix stabilization strategies, such as the use of amino acid substitutions and chemical cross-linkers (Andres, M. J. I., et al., Tetrahedron, 55:11711-11743 (1999)), can be applied to increase the helix propensity of the C-peptide.

Comparison to other studies. The C34-GNC4 heterodimer recovers almost the full biological activity of C34 (within an order of magnitude), which has been difficult to achieve in previous designed proteins using the epitope transfer strategy (22, 24). This is likely due to the ability to produce a more accurate three-dimensional reproduction of the native binding epitope in the protein scaffold than in previous designs. However, the structural similarity between the C34 helix and the GCN4 leucine zipper helix is comparable to the superposition between the native binding epitope and the protein scaffold in previous studies. Another possible explanation involves the unusually large number of residues of the C34 binding epitope that was transferred (15 surface mutations were made to the GCN4 leucine zipper for a total of 19 residues that make up the C34 binding epitope). The transfer of an extended binding interface is likely to play a role in the increased inhibitory potency of the C34-GNC4 heterodimer, as interactions along the entire groove were found to be important for the stability of the six-helix bundle (Lu, M., et al., J. Virol., 75(22):11146-11156 (2001)). The transfer of such a large number of interfacial residues is made possible in this system partly by the fact that the C34 binding epitope is not extremely hydrophobic (FIG. 1B), allowing for solubility at their inhibitory concentrations. The inhibition data indicates that at nanomolar concentration, a substantial population is in an active state and not in an aggregate.

The role of transferring an extended interface in determining the biological activity of the mimic is shown in a comparison to previous studies, where a relatively small number of residues in the epitope were transferred, coupled with a modest recovery of biological activity. For example, 8 critical binding residues of IL-4 (a four-helix bundle) were transferred to the GCN4 leucine zipper to produce a peptide that binds to the IL-4 receptor (Domingues, H., et al., Nat. Struct. Biol., 6(7):652-656 (1999)). The best binder exhibited a K_{d} of 5.0 μM, compared to 1.4 nM for the native ligand, IL-4. In another study, the CDR2-like loop of the CD4 receptor, which is crucial in CD4 binding to HIV gp120, was reproduced onto a structurally homologous β-hairpin region of a small stable protein scaffold, scyalltoxin (Vita, C., et al., Proc. Natl. Acad. Sci., USA, 96(23):13091-13096 (1999)). The best mimic, which contained 10 transferred residues and 2 additional mutations, binds to gp 120 with affinities of 0.1 to 1.0 μM, about 100 times weaker than the native ligand, soluble CD4. Attempts to transfer a larger number of residues in these studies were hampered by the stability of the scaffold protein. For the IL-4 mimic, NMR spectroscopy indicated that the GCN4 leucine zipper was partially unfolded (Domingues, H., et al., Nat. Struct. Biol., 6(7):652-656 (1999)), despite a smaller number of transferred residues than in the current C-peptide study. This may have been due to mutations to the e and g positions of the GCN4 portion of the IL-4 mimic; the e and g positions are unchanged in the GCN4 portion of the C34-GNC4 heterodimers. In addition, an attempt to transfer a larger number of DNA-binding residues to a solvent-exposed face of a two-helix bundle resulted in the destruction of the native fold of the protein scaffold (Chin, J. W., et al., J. Am. Chem. Sco., 123:2929-2930 (2001); Zondio, N. J., et al., J. Am. Chem. Soc., 121:6938-6939 (1999)). In that system, combinatorial selections was used to find compensatory mutations in the hydrophobic core which restored the native protein fold and resulted in significant improvement in DNA-binding activity ((Chin, J. W., et al., J. Am. Chem. Sco., 123.2929-2930 (2001)).

Besides transferring a large binding epitope, the procedures can be made more efficient by transferring only the critical residues in the binding interface that contribute the most binding energy. For example, in some cases, the majority of the binding energy stems from a few strong interactions (“hot spots”) (Clackson, T., et al., Science, 267(5196).383-386 (1995)) involving residues that are generally solvent-accessible, conformationally adaptive and non-polar (DeLano, W. L., et al., Science, 287:1279-1283 (2000)). In separate studies using relaxation measurements by nuclear magnetic resonance spectroscopy, the backbones of the most critical binding residues were found to be conformationally flexible in the unbound state (Feher, V. A., et al., Nature, 400:289-293 (1999)), and the side chains of the most critical binding residues were highly restricted in the bound state (Kay, L. E., Nat. Struct. Biol., 5(2):156-163 (1998)). The identification of energetically critical binding residues can help minimize the number of total residues transferred, and thereby minimize disturbance to the protein scaffold. Combinatorial selection strategies (Chin, J. W., et al., J. Am. Chem. Soc., 123:2929-2930 (2001); Sidhu, S. S., et al., Methods Enzymol., 328:333-336 (2000)) and computational approaches (Ghirlanda, G., et al., J. Mol. Biol., 281:379-391 (1998); Dahiyat, B. I., et al., Prot. Sci, 5:895-903 (1996); Koehl, P., et al., J. Mol. Biol., 239:249-275 (1994); Harbury, P. B., et al., Science, 282:1462-1467 (1998)) could also be used to optimize the binding epitope after the initial transfer.

Future design. The C34-GNC4 heterodimer involves the oxidation of two cysteine residues, linking two separate peptides together to form a covalent heterodimer. This step can be eliminated by engineering a single chain variant, allowing for recombinant expression and therefore bypassing the need for chemical synthesis. For example, an antiparallel coiled coil instead of a parallel coiled coil can be used (Oakley, M. G., et al., Biochemistry, 37:12603-12610 (1998); Zuccola, H. J., et al., Structure, 6(7):821-830 (1998)). Also, the use of higher order GCN4-derived oligomers as protein scaffolds will likely yield binders of higher affinity; since the structures of GCN4-derived trimers and tetramers exhibit less supercoil than the GCN4 dimer, the binding epitope would require less conformational change to adopt standard helical conformations.

Other viruses. The trimer-of-hairpins motif is used for membrane fusion in many viruses, including retroviruses, orthoviruses, paramyxoviruses and filoviruses (Singh, M., et al., J. Mol. Biol., 290(5):1031-1041 (1999); Weissenhom, W., et al., Mol. Cell., 2(5):605-616 (1998); Malashkevich, V. N., et al., Proc. Natl. Acad. Sci., USA, 96(6):2662-2667 (1999); Malashkevich, V. N., et al., Proc. Natl. Acad. Sci., USA, 98(15):8502-8506 (2001); Zhao, X., et al., Proc. Natl. Acad. Sic., USA, 97(26):14172-14177 (2000)). In some of these cases, analogous “C-peptides” have been shown to inhibit membrane fusion (Lambert, D. M., et al., Proc. Natl. Acad. Sci., USA, 93:2186-2191 (1996); Yao, Q., et al., Virology, 223:103-112 (1996); Rapaport, D., et al., EMBO J., 14(22):5524-5531 (1995)). The strategy of epitope transfer described in this report can be adopted for these other viruses as well. Finally, this strategy is likely effective for conferring stable helical structures onto other biologically active peptides (Zutshi, R., et al., Curr. Opin. Chem. Biol., 2:62-66 (1998); Peczuh, M. W., et al., Chem. Rev., 100:2479-2494 (2000)).

While this invention has been particularly shown and described with references to preferred embodiments thereof, it will be understood by those skilled in the art that various changes in form and details may be made therein without departing from the scope of the invention encompassed by the appended claims.

What is claimed is:

1. A structured C-peptide comprising all or a portion of a C-peptide from a virus and a polypeptide scaffold.

2. The structured C-peptide of claim 1, comprising all or a portion of HIV-1 gp41 C34 peptide and a polypeptide scaffold which is a GCN4 leucine zipper or a portion thereof.

3. A structured C-peptide comprising a binding epitope of HIV-1 C34 linked to a GCN4 leucine zipper, wherein the structured C-peptide is stable, helical and biologically active.

4. The structured C-peptide of claim 3 which further comprises a second GCN4 leucine zipper linked to the structured C-peptide via a disulfide bond.

5. The structured C-peptide of claim 4 wherein the disulfide bond occurs between two cysteine residues.

6. A structured C-peptide comprising a first peptide and a second peptide, the first peptide comprising all or a portion of a C-peptide from a virus grafted onto the surface of a GCN4 leucine zipper and the second peptide comprising an additional GCN4 peptide, wherein the first and second peptide are linked and the structured C-peptide is stable, helical and biologically active.

7. The structured C-peptide of claim 6 wherein the C-peptide from a virus is a binding epitope of HIV-1 C34.

8. The structured C-peptide of claim 7 wherein the first peptide and the second peptide are linked via a disulfide bond between two cysteine residues.

9. A structured C-peptide comprising a first peptide and a second peptide, the first peptide comprising all or a portion of a binding epitope of HIV-1 C34 grafted onto the surface of a GCN4 leucine zipper and the second peptide comprising an additional GCN4 peptide, wherein the first and second peptide are linked and the structured C-peptide is stable, helical and biologically active.

10. The structured C-peptide of claim 9 wherein the first peptide and the second peptide are linked via a disulfide bond between two cysteine residues.

11. A structured C-peptide comprising a first peptide and a second peptide, the first peptide comprising all or a portion of a binding epitope of HIV-1 C34 grafted onto the surface of a GCN4 leucine zipper and the second peptide comprising an additional GCN4 peptide, wherein the first and second peptide are linked, Asn 16 in each GCN4 peptide is replaced with Lys, and the structured C-peptide is stable, helical and biologically active.

12. The structured C-peptide of claim 11 wherein the first peptide and the second peptide are linked via a disulfide bond between two cysteine residues.

13. A structured C-peptide comprising C34-GCN4.

14. A structured C-peptide comprising C34 coil.

15. A structured C-peptide comprising C34-GCN4-N16K.

16. A method of producing a structured viral C-peptide that is stable, helical and biologically active comprising:
a) grafting a first peptide comprising all or a portion of a C-peptide from a virus onto the surface of a GCN4 leucine zipper; and
b) linking a second peptide comprising an additional GCN4 leucine zipper to the first peptide,
thereby producing a structured viral C-peptide that is stable, helical and biologically active.

17. A method of eliciting an immune response in an individual, comprising introducing into the individual a structured C-peptide.

18. A method of eliciting an immune response to a virus in an individual, comprising introducing into the individual a structured C-peptide comprising all or a portion of a C-peptide from a virus and a polypeptide scaffold.

19. A method of eliciting an immune response to HIV in an individual, comprising introducing into the individual a structured C-peptide comprising a binding epitope of HIV-1 C34 linked to a GCN4 leucine zipper, wherein the structured C-peptide is stable, helical and biologically active.

20. The method of claim 19 wherein the structured C-peptide comprises a first peptide and a second peptide, the first peptide comprising all or a portion of a binding epitope of HIV-1 C34 grafted onto the surface of a GCN4 leucine zipper and the second peptide comprising an additional GCN4 leucine zipepr, wherein the first and second peptide are linked and the structured C-peptide is stable, helical and biologically active.

21. The method of claim 20 wherein Asn 16 in each GCN4 peptide is replaced with Lys.

22. A method of inhibiting entry of HIV into cells of an individual comprising introducing into the individual a structured C-peptide comprising a first peptide and a second peptide, the first peptide comprising all or a portion of a binding epitope of HIV-1 C34 grafted onto the surface of a GCN4 leucine zipper and the second peptide comprising an additional GCN4 leucine zipper, wherein the first and second peptide are linked and the structured C-peptide is stable, helical and biologically active.

23. A method of identifying a drug that binds to an amino terminal region of HIV gp41 comprising:
a) combining
i) the amino-terminal region of gp41,
ii) a structured C-peptide comprising a first peptide and a second peptide, the first peptide comprising all or a portion of a binding epitope of HIV-1 C34 grafted onto the surface of a GCN4 leucine zipper and the second peptide comprising an additional GCN4 leucine zipper, wherein the first and second peptide are linked and the structured C-peptide is stable, helical and biologically active, and
iii) a candidate drug
thereby producing a combination;
b) maintaining the combination under conditions appropriate for binding of the structured C-peptide to the amino terminal region of gp41;
c) determining the extent to which the structured C-peptide binds to the amino terminal region of gp41 in the presence of the candidate drug compared to a control sample,
wherein if the binding of the structured C-peptide to the amino terminal region of gp41 occurs to a lesser extent in the presence of the candidate drug compared to a control sample, then the drug binds to an amino terminal region of HIV gp41.


SEQUENCE LISTING (13 sequences)

SEQ ID NO: 1 - protein, 33 aa, Unknown
  GCN4 peptide
RMKQLEDKVEELLSKNYHLENEVARLKKLVGER

SEQ ID NO: 2 - protein, 34 aa, Unknown
  C34 peptide
WMEWDREINNYTSLIHSLIEESQNQQEKNEQELL

SEQ ID NO: 3 - protein, 34 aa, Unknown
  (Cys) C34-GCN4 peptide
WRMWDLEIKVYTLLIKNLILESEVQQLKNLVELR

SEQ ID NO: 4 - protein, 36 aa, Unknown
  CGG-GCN4 peptide
CGGRMKQLEDKVEELLSKNYHLENEVARLKKLVGER

SEQ ID NO: 5 - protein, 37 aa, Unknown
  CGG-C34-GCN4 peptide
CGGWRMWDLEIKVYTLLIKNLILESEVQQLKNLVELR

SEQ ID NO: 6 - protein, 36 aa, Unknown
  CGG-GCN4-N16K peptide
CGGRMKQLEDKVEELLSKKYHLENEVARLKKLVGER

SEQ ID NO: 7 - protein, 37 aa, Unknown
  CGG-C34-GCN4-N16K peptide
CGGWRMWDLEIKVYTLLIKKLILESEVQQLKNLVELR

SEQ ID NO: 8 - protein, 36 aa, Unknown
  CGG-GCN4-pIL peptide
CGGRMKQLEDKIEELLSKIYHLENEIARLKKLIGER

SEQ ID NO: 9 - protein, 37 aa, Unknown
  CGG-C34-GCN4-pIL peptide
CGGWRMWDLEIKIYTLLIKILILESEIQQLKNLIELR

SEQ ID NO: 10 - protein, 4 aa, Unknown
  Four amino acids of overlap between C34 and GCN4 of the C34-GCN4 heterodimer
ELEK

SEQ ID NO: 11 - protein, 44 aa, Unknown
  C34 and 2F5 epitope
WMEWDREINNYTSLIHSLIEESQNQQEKNEQELLELDKWASLWN

SEQ ID NO: 12 - protein, 6 aa, Unknown
  Core epitope for the 2F5 monoclonal antibody
ELDKWA

SEQ ID NO: 13 - protein, 40 aa, Unknown
  C34-2F5 eptiope
WMEWDREINNYTSLIHSLIEESQNQQEKNEQELLELDKWA